(19) United States
(12) Patent Application Publication
(10) Pub. No.: US 2004/0265814 A1
(43) Pub. Date: Dec. 30, 2004

(54) Method for detecting cytosine methylation by comparatively analysing single strands of amplificates

(76) Inventors: Jurgen Distler, Berlin (DE); Erik Leu, Berlin (DE)

Correspondence Address: KRIEGSMAN & KRIEGSMAN, 665 FRANKLIN STREET, FRAMINGHAM, MA, 01702, US

(21) Appl. No.: 10/482,433

(22) PCT Filed: Jun. 27, 2002
(86) PCT No.: PCT/DE02/02433

(30) Foreign Application Priority Data
Jun. 27, 2001 (DE) 10132212.7

(51) Int. Cl. C12Q 1/68; C12P 19/34

(52) U.S. Cl. 435/6; 435/91.2

(57) ABSTRACT

A method is described for the detection of cytosine methylation in DNA samples. A genomic DNA sample is chemically treated, preferably with a bisulfite (=disulfite, hydrogen sulfite), in such a way that cytosine is converted to uracil, while 5-methylcytosine remains unchanged. Segments of the sample DNA are amplified with at least 2 primers in a polymerase reaction, preferably a polymerase chain reaction. Finally, the fragments are investigated with respect to the base composition of each of the two complementary strands of the amplificate, whereby a conclusion is made on the methylation state in the amplified segment of the genomic DNA sample from the difference in molecular weight of the two strands.

The present invention concerns a method for the detection of cytosine methylation in DNA samples.

The levels of observation that have been well studied in molecular biology according to developments in methods in recent years include the genes themselves, the transcription of these genes into RNA and the proteins forming therefrom. During the course of development of an individual, which gene is turned on and how the activation and inhibition of certain genes in certain cells and tissues are controlled can be correlated with the extent and nature of the methylation of the genes or of the genome. In this regard, pathogenic states are also expressed by a modified methylation pattern of individual genes or of the genome.

5-Methylcytosine is the most frequent covalently modified base in the DNA of eukaryotic cells. For example, it plays a role in the regulation of transcription, in genetic imprinting and in tumorigenesis. The identification of 5-methylcytosine as a component of genetic information is thus of considerable interest. 5-Methylcytosine positions, however, cannot be identified by sequencing, since 5-methylcytosine has the same base-pairing behavior as cytosine. In addition, in the case of a PCR amplification, the epigenetic information which is borne by the 5-methylcytosines is completely lost.

A relatively new method that in the meantime has become the most widely used method for investigating DNA for 5-methylcytosine is based on the specific reaction of bisulfite with cytosine, which, after subsequent alkaline hydrolysis, is then converted to uracil, which corresponds in its base-pairing behavior to thymidine. In contrast, 5-methylcytosine is not modified under these conditions. Thus, the original DNA is converted so that methylcytosine, which originally cannot be distinguished from cytosine by its hybridization behavior, can now be detected by “standard” molecular biology techniques as the only remaining cytosine, for example, by amplification and hybridization or sequencing. All of these techniques are based on base pairing, which is now fully utilized. The prior art, which concerns sensitivity, is defined by a method that incorporates the DNA to be investigated in an agarose matrix, so that the diffusion and renaturation of the DNA are prevented (bisulfite reacts only on single-stranded DNA) and all precipitation and purification steps are replaced by rapid dialysis (Olek A, Oswald J, Walter J. A modified and improved method for bisulphite based cytosine methylation analysis. Nucleic Acids Res. Dec. 15, 1996; 24(24):5064-6). Individual cells can be investigated by this method, which illustrates the potential of the method. Of course, up until now, only individual regions of up to approximately 3000 base pairs long have been investigated; a global investigation of cells for thousands of possible methylation analyses is not possible. To be sure, very small fragments of small quantities of sample also cannot be reliably analyzed by this method. These are lost despite the protection from diffusion by the matrix.

An overview of other known possibilities for detecting 5-methylcytosines can be derived from the following review article: Rein T, DePamphilis M L, Zorbas H. Identifying 5-methylcytosine and related modifications in DNA genomes. Nucleic Acids Res. May 15, 1998; 26(10): 2255-64.

The bisulfite technique has been previously applied only in research, with a few exceptions (e.g., Zeschnigk M, Lich C, Buiting K, Dörfler W, Horsthemke B. A single-tube PCR test for the diagnosis of Angelman and Prader-Willi syndrome based an allelic methylation differences at the SNRPN locus. Eur J Hum Genet. March-April 1997; 5(2):94-8). However, short, specific segments of a known gene have always been amplified after a bisulfite treatment and either completely sequenced (Olek A, Walter J. The pre-implantation ontogeny of the H19 methylation imprint. Nat Genet. November 1997; 17(3): 275-6) or individual cytosine positions have been detected by a “primer extension reaction” (Gonzalgo M L, Jones P A. Rapid quantitation of methylation differences at specific sites using methylation-sensitive single nucleotide primer extension (Ms-SNuPE). Nucleic Acids Res. Jun. 15, 1997; 25(12): 2529-31, WO Patent 95-00669) or an enzyme step (Xiong Z, Laird P W. COBRA: a sensitive and quantitative DNA methylation assay. Nucleic Acids Res. Jun. 15, 1997; 25(12): 2532-4). Detection by hybridization has also been described (Olek et al., WO 99-28498).

One problem of the bisulfite reaction is that it frequently runs incompletely. This means that unconverted cytosines may indicate not only that they are methylated, but also an incomplete bisulfite reaction. It is therefore of great interest to be able to quantitatively follow the bisulfite reaction and to be able to determine its success prior to the determination of the methylation degree. Approaches to this by means of bisulfite sequencing have been published (Grunau, C., Rosenthal, A. Bisulfite genomic sequencing: systematic investigation of critical experimental parameters. Nucleic Acids Res. Jul. 1, 2001; 29(13): E65-5). This method, however, is tedious and expensive and very large quantities of sample are required, so that it is less suitable for routine application.

Urea improves the efficiency of the bisulfite treatment prior to the sequencing of 5-methylcytosine in genomic DNA (Paulin R, Grigg G W, Davey M W, Piper A A. Urea improves efficiency of bisulphate*-mediated sequencing of 5-methylcytosine in genomic DNA. Nucleic Acids Res. Nov. 1, 1998; 26(21): 5009-10). *sic; bisulphite?—Trans Note

Other publications which are concerned with the application of the bisulfite technique to the detection of methylation in the case of individual genes are: Grigg G, Clark S. Sequencing 5-methylcytosine residues in genomic DNA. Bioassays**. June 1994; 16(6): 431-6, 431; Zeschnigk M, Schmitz B, Dittrich B, Buiting K, Horsthemke B, Dörfler W. Imprinted segments in the human genome: **sic; Bioessays?—Trans. Note different DNA methylation patterns in the Prader-Willi/Angelman syndrome region as determined by the genomic sequencing method. Hum Mol Genet. March 1997; 6(3): 387-95; Feil R, Charlton J, Bird A P, Walter J, Reik W. Methylation analysis on individual chromosomes: improved protocol for bisulphate* genomic sequencing. Nucleic Acids Res. February 1994 25; 22(4): 695-6; Martin V, Ribieras S, Song-Wang X, Rio M C, Dante R. Genomic sequencing indicates a correlation between DNA hypomethylation in the 5′ region of the pS2 gene and in its expression in human breast cancer cell lines. Gene. May 19, 1995; 157(1-2): 261-4; WO 97 46705, WO 95 15373 and WO 45560. *sic; bisulphite?—Trans. Note

Another known method is the so-called methylation-sensitive PCR (Herman J G, Graff J R, Myohanen S, Nelkin B D, Baylin S B (1996), Methylation-specific PCR: a novel PCR assay for methylation status of CpG islands. Proc Natl Acad Sci U S A. September 3; 93(18): 9821-6). For this method, primers are used, which hybridize either only to a sequence that forms by the bisulfite treatment of a DNA which is unmethylated at the respective position, or, vice versa, primers which bind only to a nucleic acid which forms by the bisulfite treatment of a DNA unmethylated** at the respective position. Amplificates can be produced accordingly with these primers, the detection of which in turn supplies indications of the presence of a methylated or unmethylated position in the sample to which the primers bind. **sic; methylated?—Trans. Note

A newer method is also the detection of cytosine methylation by means of a Taqman PCR, which has become known as “methyl light” (WO 00/70090). It is possible with this method to detect the methylation state of individual positions or a few positions directly in the course of the PCR, so that a subsequent analysis of the products becomes superfluous.

The prior art is again a method developed by Epigenomics, which amplifies DNA to be investigated and background DNA to the same extent after bisulfite treatment and then the former CpG positions that are contained in the fragment are investigated by hybridization techniques, or alternatively by means of minisequencing or other current methods. This has the advantage that one obtains a quantitative pattern with respect to the investigated methylation positions, i.e., it produces a determination of the degree of methylation from a plurality of positions, which makes possible a very precise classification, e.g., in the case of solid tumors.

Primer oligonucleotides with multiple fluorescent labels have been used for the labeling of amplificates. Particularly suitable for fluorescent labels is the simple introduction of Cy3 and Cy5 dyes at the 5′-end of the respective primer. The dyes Cy3 and Cy5, in addition to many others, are commercially available.

Matrix-assisted laser desorptions/ionization mass spectrometry (MALDI-TOF) is a very powerful development for the analysis of biomolecules (Karas M, Hillenkamp F. Laser desorption ionization of proteins with molecular masses exceeding 10,000 daltons. Anal Chem. Oct. 15, 1988; 60(20): 2299-301). An analyte is embedded in a light-absorbing matrix. The matrix is vaporized by a short laser pulse and the analyte molecule is transported unfragmented into the gas phase. The analyte is ionized by collisions with matrix molecules. An applied voltage accelerates the ions in a field-free flight tube. Ions are accelerated to varying degrees based on their different masses. Smaller ions reach the detector sooner than larger ones.

MALDI-TOF spectroscopy is excellently suitable for the analysis of peptides and proteins. The analysis of nucleic acids is somewhat more difficult (Gut, I. G. and Beck, S. (1995), DNA and Matrix Assisted Laser Desorption Ionization Mass Spectrometry. Molecular Biology: Current Innovations and Future Trends 1: 147-157). For nucleic acids, the sensitivity is approximately 100 times poorer than for peptides and decreases overproportionally with increasing fragment size. For nucleid acids, which have a multiply negatively charged backbone, the ionization process via the matrix is essentially less efficient. In MALDI-TOF spectroscopy, the choice of matrix plays an imminently important role. Several very powerful matrices, which produce a very fine crystallization, have been found for the desorption of peptides. In the meantime, several effective matrices have been developed for DNA, but the difference in sensitivity has not been reduced thereby. The difference in sensitivity can be reduced by modifying the DNA chemically in such a way that it resembles a peptide. Phosphorothioate nucleic acids, in which the usual phosphates of the backbone are substituted by thiophosphates, can be converted by simple alkylation chemistry into a charge-neutral DNA (Gut, I. G. and Beck, S. (1995), A procedure for selective DNA alkylation and detection by mass spectrometry. Nucleic Acids Res. 23: 1367-1373). The coupling of a “charge tag” to this modified DNA results in an increase in sensitivity to the same amount as is found for peptides. Another advantage of “charge tagging” is the increased stability of the analysis in the presence of impurities, which make the detection of unmodified substrates very difficult.

Genomic DNA is obtained from DNA of cells, tissue or other test samples by standard methods. This standard methodology is found in references such as Fritsch and Maniatis, Molecular Cloning: A Laboratory Manual, 1989.

After the invention of PCR, numerous variants became known in the next few years, which refine this technique for the amplification of DNA. In particular, multiplexing of the PCR (multiplex PCR) should be mentioned here, wherein more than 2 specific primers are used and thus a plurality of different, specific amplifications can be produced in one reaction vessel. Particularly interesting also is so-called nested PCR, which is used among other things for the detection of particularly small quantities of DNA. This type of PCR consists of two amplifications, one following the other, wherein the primers of the second amplification lie within the first amplificate and are not identical to the primers of the first amplification. In this way, a particular specificity is achieved, since the primers of the second amplification only function if the intended fragment has been produced in the first amplification. In contrast, the propagation of any possible byproducts of the first amplification in the second amplification is excluded as much as possible.

Accordingly, a great many methods for methylation analysis are prior art. Most of these methods permit the analysis of individual positions in the genome; several, such as, for example, hybridization techniques on oligomer arrays, permit the simultaneous analysis of a plurality of positions. The experimental cost of these methods, however, is comparatively high. The present invention will provide a method, which, after the bisulfite treatment and amplification, by employing widely used equipment in molecular biology laboratories, such as capillary gel electrophoresis or HPLC, permits conducting a direct methylation analysis of the entire fragment without further steps. The method thus relinquishes the analysis of specific individual positions, but rather analyzes the extent of methylation in the investigated fragment.

The present invention is thus based on the knowledge that the base composition of the DNA in the bisulfite treatment and in the subsequent amplification changes in a characteristic way and that an analytical method for the detection of cytosine methylation can be derived from this alone. If a genomic DNA is treated with bisulfite, then all unmethylated cytosine bases are converted to uracil and then to thymine in the following amplification. Consequently, the number of cytosine bases essentially decreases and, in fact, this occurs to a greater extent, the smaller the degree of methylation of the respective amplified segment of the DNA sample. The number of thymine bases increases correspondingly, the smaller the methylation degree. In the complementary counterstrand formed in the amplification, on the other hand, the smaller the methylation degree in the DNA sample, then the more adenine is incorporated. In contrast, the counterstrand contains more guanine, the higher the methylation degree of the DNA sample.

This now produces the effect that the molecular mass of the two complementary strands formed in the amplification differs to a greater extent, the smaller the methylation degree in the amplified segment of the genomic DNA sample. A conversion of the cytosine in one of the strands finally to thymidine leads to an increase in the mass by 15 Da each time, while on the complementary strand it results that correspondingly guanine is replaced by adenine, and a reduction of the molecular mass by 16 Da results. It follows from this that a difference in mass of 31 Da between the two complementary strands of the amplificate results from the conversion of each additional cytosine finally to thymine.

The present invention now utilizes several methods for indicating this difference in mass and to directly derive therefrom information on the methylation state of the investigated segment of the genomic DNA sample.

In addition to the molecular mass of the individual strands, which changes increasingly with decreasing methylation, other effects occur, which can be utilized here. Each conversion of a cytosine base to thymidine, for example, leads to the loss of an amino function in the respective single strand, while in the other strand, guanine is exchanged for adenine and thus the amino function remains. The properties of the respective single strands thus change considerably relative to one another, which can be exploited by the selected analytical method. In each case, however, there is a direct dependence of these properties on the methylation degree.

Several methods, which will be described here, can be used for the analysis of individual strands. In particular, denaturing gel electrophoresis, preferably capillary gel electrophoresis, is suitable for the separation of the individual strands (see Example 1). Normally, in gel electrophoresis, if it is not conducted in a denaturing manner, the DNA is essentially separated on the basis of length. Thus DNA fragments of known length serve as a standard. In the case of denaturing gel electrophoresis, in contrast, there often occurs also a separation as a function of sequence, if different sequences induce different conformations and secondary structures of the DNA single strand. One of the most well-known techniques in this connection is SSCP.

However, since here a methylation degree is to be established within a fragment, which may have a plurality of possible different sequences after the bisulfite treatment, such methods are not very suitable, since one would have to distinguish between very many different cases. An application of SSCP, however, is also conceivable for methylation analysis in this sense.

The particular advantage of this invention with respect to gel electrophoresis, however, is that the base composition in the bisulfite-treated and amplified DNA does not substantially differ from the genomic DNA, and, in fact, this is more pronounced, the smaller its methylation degree. These differences are quite sufficient so that they can be used directly for methylation analysis, as shown in Example 1, since the behavior in capillary gel electrophoresis changes so that it can be measured as a function of the sequence. It is particularly meaningful and preferred also to use the distance between the bands for the two respective single strands of the PCR product as a measure for the methylation degree in the genomic sample.

A similar consideration also applies to the two peaks of a denaturing HPLC, which can be evaluated analogously. The two single strands can be separated by elution on suitable reversed-phase columns, preferably in combination with triethylammonium acetate/acetonitrile gradients. Here also, the retention time is directly dependent on the base composition, and thus finally on the methylation degree of the genomic DNA sample in the fragment in question.

It is also possible and preferred to conduct the HPLC at a temperature at which the DNA is present at least partially still in double-stranded form. The duplexes and heteroduplexes formed in this way can also be separated as a function of the number of erroneous pairings by HPLC. This permits generating an image of the homogeneity of the methylation between two samples. It is also possible and preferred to measure methylation directly in this way, when a known reference amplificate is added, which has been obtained from a sample that has been well characterized and treated with bisulfite. The peaks in this case permit a conclusion on the similarity of the methylation pattern to that of the reference DNA.

This object that is the basis of the invention is solved by creating a method for the detection of cytosine methylation in DNA samples, in which the following steps are conducted:

a) a genomic DNA sample is chemically treated, preferably with a bisulfite (=disulfite, hydrogen sulfite), in such a way that cytosine is converted into uracil, while 5-methylcytosine remains unchanged;

b) segments of the sample DNA are amplified with at least 2 primers in a polymerase reaction, preferably a polymerase chain reaction and

c) the fragments are investigated with respect to the base composition of each of the two complementary strands of the amplificate, whereby a conclusion is made on the methylation state in the amplified segment of the genomic DNA sample from the difference in molecular weight of the two strands.

In a particularly preferred variant of the method, the difference or the differences in molecular weight of the two strands are measured by denaturing gel electrophoresis. The empirical composition of the DNA can be considered analogously to the molecular weight with respect to the nucleobases A, C, T and G. In the following, however, reference is made only to the molecular weight for reasons of simplicity. In an again particularly preferred variant of the method, the difference in molecular weight of the two strands is determined by capillary gel electrophoresis.

In another particularly preferred variant of the method, the difference in molecular weight of the two strands is measured by a chromatographic method. This chromatographic method particularly preferably involves denaturing high-performance liquid chromatography (HPLC).

In addition to molecular weight, still other factors, such as, e.g., the different total content of guanine, amino functions or keto functions of the two complementary strands particularly preferably also contribute to their different behavior in one of the above-mentioned analytical methods.

It is also particularly preferred to determine the difference in the molecular weight of the two strands by mass spectrometry. Since the mass difference of the two strands is determined exclusively, a calibration becomes superfluous here. It is obvious, however, that it is also possible to determine the masses of the two strands separately and to make use of only one mass for the methylation analysis.

A method variant is also particularly preferred, in which reference DNA of known composition and identical or similar length is used in the analysis as the external or internal standard. This reference DNA again particularly preferably involves bisulfite-treated DNA composed of a reference sample with known methylation state or the amplified genomic DNA with a fragment length identical or similar to each analyzed fragment without prior chemical treatment. This method variant is preferably conducted with small sample volumes, but is suitable also for mass throughput.

In another particularly preferred variant of the method, the quality and completeness of the bisulfite reaction is monitored at the same time as the analysis of the methylation state. The bisulfite reaction generally does not take place when the DNA to be treated is not single-stranded. In the case of incomplete denaturing, a fraction of the DNA can remain practically completely unconverted. Depending on the specificity of the primers, they can be used for the amplification of unconverted DNA and residues of practically genomic DNA. These genomic amplificates can be detected simultaneously with the analysis of the methylation state, since fragments are also observed with approximately average base composition and thus approximately the expected mass (Example 4). It is also particularly preferred to use primers which amplify the bisulfite-converted DNA and the genomic DNA to the same extent, in order to be able to also detect small quantities of unconverted DNA in this way.

A method is thus particularly preferred, in which the quality of the bisulfite reaction and the methylation degree are measured at the same time by also detecting unconverted fractions. This is preferably achieved by the fact that the primers used amplify bisulfite-converted DNA and genomic DNA to the same extent.

In a particularly preferred variant of the method, the DNA samples are obtained from serum or other body fluids of an individual, from cell lines, blood, sputum, stool, urine, serum, cerebrospinal fluid, tissue embedded in paraffin, for example, tissue from eyes, intestine, kidney, brain, heart, prostate, lung, breast or liver, histological slides and all possible combinations thereof.

In another particularly preferred variant of the method, the chemical treatment is conducted with a bisulfite (=disulfite, hydrogen sulfite). The chemical treatment is particularly preferably conducted after embedding the DNA in agarose. It is also preferred that in the chemical treatment, a reagent that denatures the DNA duplex and/or a radical trap is/are present.

In a particularly preferred method variant, the amplification of several fragments is conducted in one reaction vessel in the form of a multiplex PCR.

The primers used in the amplifications do not most preferably amplify fragments of genomic DNA that is not treated with bisulfite (or only do so to a negligibly small extent), so that they are specific for the DNA converted with bisulfite. This protects from erroneous results in the case of an incomplete conversion reaction with sodium bisulfite, for example, but also does not permit detection of the quality of the bisulfite reaction.

In a particularly preferred variant of the method, the amplificates are provided with at least one detectable label for detection, which is preferably introduced by labeling the primers during the amplification. The labels are most preferably fluorescent labels or radionuclides. The two strands of the amplificates are particularly preferably separated and detected as a whole in the mass spectrometer and thus are clearly characterized by their respective mass.

A method variant is also particularly preferred in which a conclusion is made on the presence of a disease or another medical condition of the patient from the methylation degree of the different CpG positions investigated.

The subject of the present invention is also the use of one of the described method variants for the diagnosis and/or prognosis of adverse events for patients or individuals, whereby these adverse events belong to at least one of the following categories: undesired drug interactions; cancer diseases; CNS malfunctions, damage or disease; symptoms of aggression or behavioral disturbances; clinical, psychological and social consequences of brain damage; psychotic disturbances and personality disorders; dementia and/or associated syndromes; cardiovascular disease, malfunction and damage; malfunction, damage or disease of the gastrointestinal tract; malfunction, damage or disease of the respiratory system; lesion, inflammation, infection, immunity and/or convalescence; malfunction, damage or disease of the body as [a consequence of] an abnormality in the development process; malfunction, damage or disease of the skin, the muscles, the connective tissue or the bones; endocrine and metabolic malfunction, damage or disease; headaches or sexual malfunction.

The subject of the present invention is also the use of one of the described method variants for distinguishing cell types or tissues or for investigating cell differentiation.

The subject of the present invention is also a kit consisting of a reagent containing bisulfite, primers for producing the amplificates, as well as, optionally, instructions for conducting an assay corresponding to one of the described method variants.

The following examples explain the invention.

### EXAMPLE 1

### Amplification of Fragments of the Mdrl Gene by PCR (Genomic DNA)

Human DNA (Promega) was used for the experiments. The Mdrl fragment was amplified with the PCR primers:

CMGCATGCTGMGAAAGACCACTGCAG (SEQ. ID 1) and

TGGGMCTGTCCCATAGTAGCTCCCAGC (SEQ. ID 2) under the following reaction conditions:

1 μl Promega_DNA (2 ng/μl)

0.2 μl Taq Polymerase (5 U/μl)

0.2 μl dNTPs (final concentration 200 μM each)

1 μl dATP fluorescein-labeled (0.5 μM final concentration)

2.5 μl 10× PCR buffer (Qiagen)

2 μl Primers (SEQ. ID 1 and 2) 25 pmol/μl each

18.1 μl Water

The amplification was conducted in a PCR thermocycler (Eppendorf) with the use of the following program:

| | | |
|---|---|---|
| Step 1 | 15 min | 95° C. |
| Step 2 | 1 min | 95° C. |
| Step 3 | 45 sec | 61° C. |
| Step 4 | 1 min 15 sec | 72° C. |
| Step 5 | GOTO Step 2 | (39×) |
| Step 6 | 10 min | 72° C. |
| Step 7 | HOLD | 4° C. |

The Mdrl PCR product produced in this way has a length of 633 bp and the sequence:

| | |
|---|---|
| CAAGCATGCTGAAGAAAGACCACTGCAGAAAAATTTCTCCTAGCCTTTTCAA | (SEQ. ID: 3) |
| AGGTGTTAGGAAGCAGAAAGGTGATACAGAATTGGAGAGGTCGGAGTTTTT | |
| GTATTAACTGTATTAAATGCGAATCCCGAGAAAATTTCCCTTAACTACGTCCT | |
| GTAGTTATATGGATATGAAGACTTATGTGAACTTTGAAAGACGTGTCTACATA | |
| AGTTGAAATGTCCCCAATGATTCAGCTGATGCGCGTTTCTCTACTTGCCCTT | |
| TCTAGAGAGGTGCAACGGAAGCCAGAACATTCCTCCTGGAAATTCAACCTG | |
| TTTCGCAGTTTCTCGAGGAATCAGCATTCAGTCAATCCGGGCCGGGAGCAG | |
| TCATCTGTGGTGAGGCTGATTGGCTGGGCAGGAACAGCGCCGGGGCGTGG | |
| GCTGAGCACAGCCGCTTCGCTCTCTTTGCCACAGGAAGCCTGAGCTCATTC | |
| GAGTAGCGGCTCTTCCAAGCTCAAAGAAGCAGAGGCCGCTGTTCGTTTC | |
| CTTTAGGTCTTTCCACTAAAGTCGGAGTATCTTCTTCCAAAATTTCACGTCTT | |
| GGTGGCCGTTCCAAGGAGCGCGAGGTAGGGGCACGCAAAGCTGGGAGCT | |
| ACTATGGGACAG TTCCCA. | |

### EXAMPLE 2

### Amplification of Bisulfite-Treated Fragments of the Mdrl Gene by PCR for the Analysis of the Methylation State

For the production of methylated human DNA, which will serve as the standard for the further investigations, 700 ng of DNA were reacted with the CpG-specific methylase Sssl (BioLabs Inc) according to the manufacturer's instructions. This methylated DNA and unmethylated human DNA were treated with bisulfite as described (Olek A, Oswald J, Walter J. A modified and improved method for bisulphite based cytosine methylation analysis. Nucleic Acids Res. Dec. 15, 1996; 24(24): 5064-6). A DNA sample was also treated with bisulfite, which was correspondingly not methylated. Of these two bisulfite-treated DNA samples, one, the bisulfite fragment corresponding to the genomic fragment, was amplified by PCR with the primers TAAGTATGTTGMGAAAGATTATTGTAG (SEQ. ID 4) and TAAAAACTATCCCATAATMCTCCCMC (SEQ. ID 5). The PCR reaction conditions were as follows:

| | |
|---|---|
| 1 μl | Bisulfite-treated DNA (10 ng/(μl) |
| 0.2 μl | Taq Polymerase (5 U/(μl) |
| 0.2 μl | dNTPs (final concentration 200 μM each) |
| 1 μl | dATP fluorescein-labeled (0.5 μM final concentration) |
| 2.5 μl | 10 × PCR buffer (Qiagen) |
| 2 μl | Primers, 5 pmol/μl each |
| 18.1 μl | Water |

The amplification was conducted in a PCR thermocycler (Eppendorf) with the use of the following program:

| | | |
|---|---|---|
| Step 1 | 15 min | 95° C. |
| Step 2 | 1 min | 95° C. |
| Step 3 | 45 sec | 55° C. |
| Step 4 | 1 min 15 sec | 72° C. |
| Step 5 | GOTO Step 2 | (39×) |
| Step 6 | 10 min | 72° C. |
| Step 7 | HOLD | 4° C. |

The Mdrl PCR product produced in this way has a length of 633 bp and the methylated variant has the following sequence:

| | |
|---|---|
| TAAGTATGTTGAAGAAAGATTATTGTAGAAAAATTTTTTTTAGTTTTTTTAAAG | (SEQ. ID: 6) |
| GTGTTAGGAAGTAGAAAGGTGATATAGAATTGGAGAGGTCGGAGTTTTTGTA | |
| TTAATTGTATTAAATGCGAATTTCGAGAAAATTTTTTTTAATTACGTTTTGTAG | |
| TTATATGGATATGAAGATTTATGTGAATTTTGAAAGACGTGTTTATATAAGTT | |
| GAAATGTTTTTAATGATTTAGTTGATGCGCGTTTTTTTATTTGTTTTTTTTAGA | |
| GAGGTGTAACGGAAGTTAGAATATTTTTTTTGGAAATTTAATTTGTTTCGTAG | |
| TTTTTCGAGGAATTAGTATTTAGTTAATTCGGGTCGGGAGTAGTTATTTGTG | |
| GTGAGGTTGATTGGTTGGGTAGGAATAGCGTCGGGGCGTGGGTTGAGTAT | |
| AGTCGTTTCGTTTTTTTTGTTATAGGAAGTTTGAGTTTATTCGAGTAGCGGTT | |
| TTTTTAAGTTTAAAGAAGTAGAGGTCGTTGTTCGTTTTTTTTAGGTTTTTTTAT | |
| TAAAGTCGGAGTATTTTTTTTTAAAATTTTACGTTTTGGTGGTCGTTTTAAGG | |
| AGCGCGAGGTAGGGGTACGTAAAGTTGGGAGTTATTATGGGATAGTTTTTA. | |

### EXAMPLE 3

### Analysis of the PCR Fragments by Capillary Electrophoresis

The DNA was separated with the capillary electrophoresis system ABI Prism 310, equipped with module GS STR POP4 (Applied Biosystems, Weiterstadt) under denaturing conditions in a capillary (length 47 cm, diameter 50 μm). Sample preparation and running conditions were performed as recommended by the equipment manufacturer. The fragment size was determined via the internal length standard ROX-1000 (Applied Biosystems).

The selected running conditions were:

| | | |
|---|---|---|
| Injection time | 2 | sec |
| Injection voltage | 3.5 | kV |
| Running voltage | 15 | kV |
| Temperature | 60° | C. |
| Running time | 45 | min. |

It was under these conditions that first the amplified genomic DNA as well as the methylated and the unmethylated and then bisulfite-treated DNA samples were measured. It was to be expected that a value which substantially corresponds to the actual length of the fragment would be measured for the statistically composed respective individual strands of the amplificate of the genomic DNA. This was confirmed: values of 630.45 bases and 632.56 bases were measured for the two single strands of the amplificate, while the theoretical value is 633 bases (FIG. 1c).

It was now expected that the values would differ greatly for the amplificates of bisulfite DNA, since the base composition of the strands differs; in particular, guanine and cytosine are no longer equally distributed. Along with the above considerations, it was also calculated that a smaller difference would result between the two strands for the methylated DNA samples than in the case of the unmethylated sample. This is confirmed by the experiment. For the unmethylated sample, values of 620.87 bases and 640.46 bases are found for the respective single strands ((FIG. 1a; 633 would be expected for a statistical distribution of the bases) and values of 622.56 bases and 640.69 bases (FIG. 1b) resulted for the methylated DNA sample. The measured difference thus corresponds to 18.13 bases for methylated DNA, and, in contrast, 19.59 bases for unmethylated DNA. This measurable difference of 1.46 can be directly drawn on for the diagnosis of the methylation state in an unknown sample.

### EXAMPLE 4

### Quality Control for the Bisulfite Reaction by Fragment Analysis

A fragment of the NME3 gene is suitable, for example, as a standard quality control for the bisulfite reaction. Of course, in practice, one must always select a gene whose methylation state could be investigated each time. Nonspecific primers, which can amplify bisulfite-treated and genomic DNA, were used for the amplification. The amplification of the fragment was conducted as in Example 1. The NME3 PCR product produced in this way has a length of 686 bp and the sequence:

| | |
|---|---|
| AAGGGAATAAAGAGAAAAGAAGTACCCAGGGTCGTGGTGTCTTTGCGCTCT | (SEQ. ID: 7) |
| GTCTTTAGGACCGGGGAGAGAAGGGCTGACGCTGTGGTCGTGGCCCTGGC | |
| CGGGGGGGCGCGGGGGGGGCGGGGTTCGGGCGGTGCGGAGCAGGGCG | |
| CCGCGTGGGTGGAACCACCTGGGCGGGTTGTGGGGGATACAGTTAGTGTC | |
| CGAGCTGCTGGAGGAGACTTGGCCTCCGCAGCTGCCCTCCGGCCCCCCAC | |
| GGCTGCCGGGTTCCGGGGTGCAAGTGAAGCAGCCTCCCCGCGGAGGCCG | |
| CAGCGCCCCGACCAGGCCTCTTTAAGCGCAGGCCCCGCCCCGGGCGCCA | |
| CCGCCCCGCCCCGCGGATCCCGCTCCCGCACCGCCATCATGATCTGCCTG | |
| GTGCTGACCATCTTCGCTAACCTCTTCCCCGCGGGTGAGCCGCGCGGCGC | |
| GGGCCGGGGGCGGGTGGCCGGTGCTGGGCCGGCCTGACGGCCCGTCCC | |
| CGCCTGCCCCGCAGCCTGCACCGGCGCACACGAACGCACCTTCCTGGCCG | |
| TGAAGCCGGACGGCGTGCAGCGGCGGCTGGTGGGCGAGATTGTGCGGCG | |
| CTTCGAGAGGAAGGGCTTCAAGTTGGTGGCGCTGAAGCTGGTGCAGGTGG | |
| GGGCGCGGTGAGCGAGCGGGGGCGCGGTGTGGGGGGAAGGGGA. | |

The following nonspecific primers were used for the amplification:

| | |
|---|---|
| AAG GGA ATA AAG AGA AAA GAA GTA | (SEQ. ID: 8) |
| and | |
| TCC CCT TCC CCC CAC A. | (SEQ. ID: 9) |

The bisulfite treatment was conducted as in the literature citation (Olek A, Oswald J, Walter J. A modified and improved method for bisulphite based cytosine methylation analysis Nucleic Acids Res. Dec. 15, 1996; 24(24): 5064-6). The subsequent amplification was conducted analogously to Example 2.

The bisulfite-treated fragment obtained has the sequence:

| | |
|---|---|
| AAGGGAATAAAGAGAAAAGAAGTATTTAGGGTCGTGGTGTTTTTGCGTTTTG | (SEQ. ID: 10) |
| TTTTTAGGATCGGGGAGAGAAGGGTTGACGTTGTGGTCGTGGTTTTGGTCG | |
| GGGGGGCGCGGGGGGGGCGGGGTTCGGGCGGTGCGGAGTAGGGCGTCG | |
| CGTGGGTGGAATTATTTGGGCGGGTTGTGGGGGATATAGTTAGTGTTCGAG | |
| TTGTTGGAGGAGATTTGGTTTTCGTAGTTGTTTTTCGGTTTTTTACGGTTGTC | |
| GGGTTTCGGGGTGTAAGTGAAGTAGTTTTTTCGCGGAGGTCGTAGCGTTTC | |
| GATTAGGTTTTTTTAAGCGTAGGTTTCGTTTCGGGCGTTATCGTTTCGTTTC | |
| GCGGATTTCGTTTTCGTATCGTTATTATGATTTGTTTGGTGTTGATTATTTTC | |
| GTTAATTTTTTTTTCGCGGGTGAGTCGCGCGGCGCGGGTCGGGGGCGGGT | |
| GGTCGGTGTTGGGTCGGTTTGACGGTTCGTTTTCGTTTGTTTCGTAGTTTGT | |
| ATCGGCGTATACGAACGTATTTTTTTGGTCGTGAAGTCGGACGGCGTGTAG | |
| CGGCGGTTGGTGGGCGAGATTGTGCGGCGTTTCGAGAGGAAGGGTTTTAA | |
| GTTGGTGGCGTTGAAGTTGGTGTAGGTGGGGGCGCGGTGAGCGAGCGGG | |
| GGCGCGGTGTGGGGGGAAGGGGA. | |

The analysis of the PCR fragments was conducted by means of capillary gel electrophoresis analogously to Example 3. It was to be expected that the deviation from the theoretical fragment length for the fragment of the NME3 gene is clearly larger, since this fragment contains many more cytosines than the average. Correspondingly, values of 670 and 708 were measured for the two single strands while the theoretical value amounts to 686. The large gap of 37 bp makes possible a simple identification of residual genomic components, which are also amplified via the nonspecific primers. An additional genomic peak (C) cannot be detected between the bisulfite-specific peaks (A and B) when there is a complete conversion.

It could be shown by this means that fragment analysis is also suitable for the quality control of the bisulfite reaction.

### LEGENDS TO FIGURES

FIGS. 2-4: left column: fragment analysis; middle column: gel image; right column: method

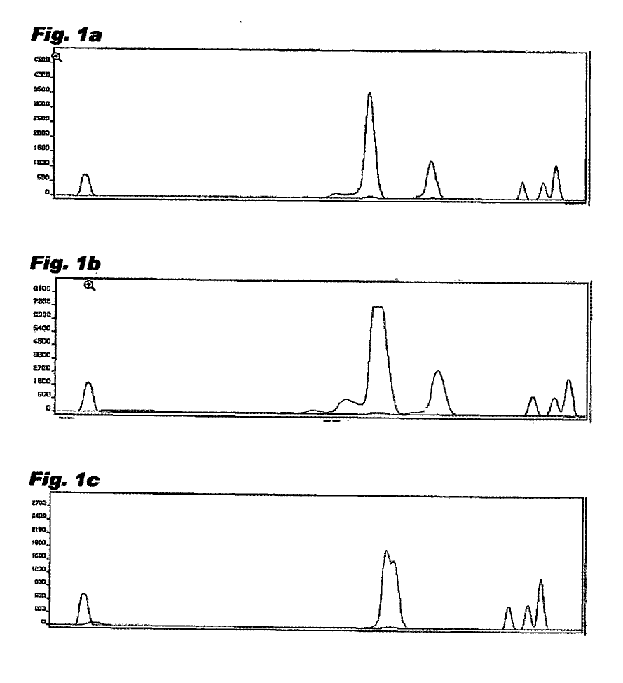

FIG. 2: Method A: Reaction temperature: 50° C.; reaction time: 5 h; thermo spikes: none; gap: 37 (671-708); residual genomic components detectable (C); Conversion incomplete

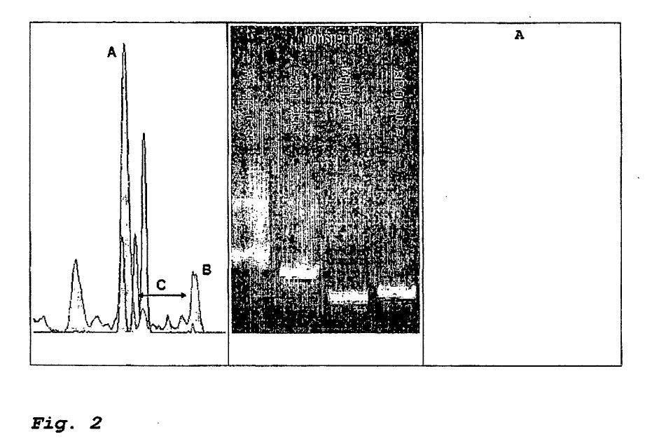

FIG. 3: Reaction temperature: 50° C.; reaction time: 2.5 h; thermo spikes: 10; splitting: 37 (671-708); residual genomic components not detectable; Conversion complete

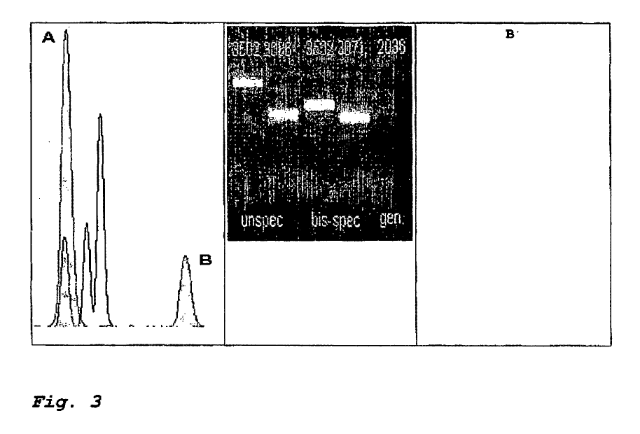

FIG. 4: Reaction temperature: 50° C.; reaction time: 5 h; thermo spikes: none; splitting: 17 (672-689); residual genomic components detectable (C); Conversion incomplete

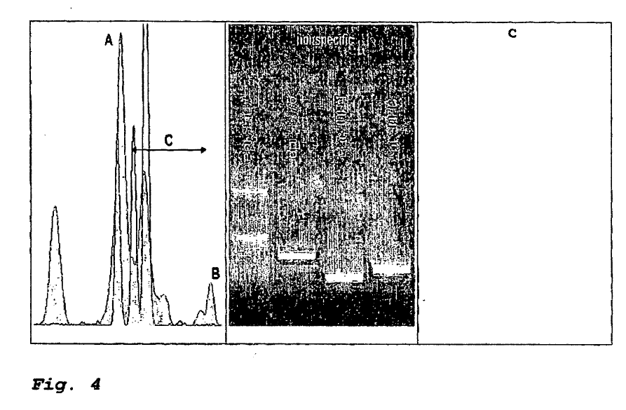

1. A method for the detection of cytosine methylation in DNA samples is hereby characterized in that the following method steps are conducted:
a genomic DNA sample is chemically treated, preferably with a bisulfite (=disulfite, hydrogen sulfite), in such a way that cytosine is converted into uracil, while 5-methylcytosine remains unchanged;
segments of the sample DNA are amplified with at least 2 primers in a polymerase reaction, preferably a polymerase chain reaction,
the fragments are investigated with respect to the base composition of each of the two complementary strands of the amplificate, whereby a conclusion is made on the methylation state in the amplified segment of the genomic DNA sample from the difference in molecular weight of the two strands.

2. The method according to claim 1, further characterized in that the difference in molecular weight of the two strands is measured by denaturing gel electrophoresis.

3. The method according to claim 1, further characterized in that the difference in molecular weight of the two strands is determined by capillary gel electrophoresis.

4. The method according to claim 1, further characterized in that the difference in molecular weight of the two strands is measured by a chromatographic method.

5. The method according to claim 1, further characterized in that denaturing high-performance liquid chromatography (HPLC) is used.

6. The method according to claim 1, further characterized in that in addition to molecular weight, still other factors, such as, e.g., the different total content of guanine, amino functions or keto functions of the two complementary strands also contribute to their different behavior in one of denaturing gel electrophoresis, capillary gel electrophoresis and a chromatographic method, such as denaturing high-performance liquid chromatography (HPLC).

7. The method according to one of the preceding claim 1, further characterized in that the difference in molecular weight of the two strands is measured by mass spectrometry.

8. The method according to one of the preceding claims claim 1, further characterized in that reference DNA of known composition and identical or similar length is used in the analysis as the external or internal standard.

9. The method according to claim 8, further characterized in that the reference DNA involves bisulfite-treated DNA composed of a reference sample with known methylation state or the amplified genomic DNA of identical or similar fragment length as each analyzed fragment, but without prior chemical treatment.

10. The method according to claim 1, further characterized in that the DNA samples are obtained from serum or other body fluids of an individual, from cell lines, blood, sputum, stool, urine, serum, cerebrospinal fluid, tissue embedded in paraffin, for example, tissue from eyes, intestine, kidney, brain, heart, prostate, lung, breast or liver, histological slides and all possible combinations thereof.

11. The method according to claim 1, further characterized in that the chemical treatment is conducted with a bisulfite (=disulfite, hydrogen sulfite).

12. The method according to claim 11, further characterized in that the chemical treatment is conducted after embedding the DNA in agarose.

13. The method according to claim 11, further characterized in that in the chemical treatment, a reagent that denatures the DNA duplex and/or a radical trap is/are present.

14. The method according to claim 1, further characterized in that the amplification of several fragments is conducted in one reaction vessel in the form of a multiplex PCR.

15. The method according to claim 1, further characterized in that the primers utilized in the amplification amplify the DNA chemically converted with bisulfite, but not the corresponding unconverted genomic sequence.

16. The method according to claim 1, further characterized in that the quality of the bisulfite reaction is measured at the same time by also detecting unconverted fractions.

17. The method according to claim 16, further characterized in that the primers used amplify bisulfite-converted DNA and genomic DNA to the same extent.

18. The method according to claim 1, further characterized in that the amplificates are provided with at least one detectable label for detection, which label is introduced preferably by labeling the primers during the amplification.

19. The method according to claim 18, further characterized in that the labels are fluorescent labels.

20. The method according to claim 18, further characterized in that the labels are radionuclides.

21. The method according to claim 1, further characterized in that the two strands of the amplificates are separated and are detected as a whole in the mass spectrometer and thus are clearly characterized by their respective mass.

22. The method according claim 1, further characterized in that a conclusion is made on the presence of a disease or another medical condition of the patient from the methylation degree of the different CpG positions investigated.

23. Use of a method according to one of the preceding claims for the diagnosis and/or prognosis of adverse events for patients or individuals, whereby these adverse events belong to at least one of the following categories: undesired drug interactions; cancer diseases; CNS malfunctions, damage or disease; symptoms of aggression or behavioral disturbances; clinical, psychological and social consequences of brain damage; psychotic disturbances and personality disorders; dementia and/or associated syndromes; cardiovascular disease, malfunction and damage; malfunction, damage or disease of the gastrointestinal tract; malfunction, damage or disease of the respiratory system; lesion, inflammation, infection, immunity and/or convalescence; malfunction, damage or disease of the body as a consequence of an abnormality in the development process; malfunction, damage or disease of the skin, the muscles, the connective tissue or the bones; endocrine and metabolic malfunction, damage or disease; headaches or sexual malfunction.

24. Use of a method according to claim 1 for the differentiation of cell types or tissues or for the investigation of cell differentiation.

25. A kit, consisting of a reagent containing bisulfite, primers for the production of amplificates, as well as, optionally, instructions for conducting an assay according to one of claims 1-22.


SEQUENCE LISTING (10 sequences)

SEQ ID NO: 1 - DNA, 28 bp, Artificial Sequence
  Primer Oligonucleotide
caagcatgctgaagaaagaccactgcag

SEQ ID NO: 2 - DNA, 28 bp, Artificial Sequence
  Primer Oligonucleotide
tgggaactgtcccatagtagctcccagc

SEQ ID NO: 3 - DNA, 633 bp, Artificial Sequence
  Amplification Product of MdRI-Fragment
caagcatgctgaagaaagaccactgcagaaaaatttctcctagccttttcaaaggtgttaggaagcagaaaggtgatacagaattggagaggtcggagtttttgtattaactgtattaaatgcgaatcccgagaaaatttcccttaactacgtcctgtagttatatggatatgaagacttatgtgaactttgaaagacgtgtctacataagttgaaatgtccccaatgattcagctgatgcgcgtttctctacttgccctttctagagaggtgcaacggaagccagaacattcctcctggaaattcaacctgtttcgcagtttctcgaggaatcagcattcagtcaatccgggccgggagcagtcatctgtggtgaggctgattggctgggcaggaacagcgccggggcgtgggctgagcacagccgcttcgctctctttgccacaggaagcctgagctcattcgagtagcggctcttccaagctcaaagaagcagaggccgctgttcgtttcctttaggtctttccactaaagtcggagtatcttcttccaaaatttcacgtcttggtggccgttccaaggagcgcgaggtaggggcacgcaaagctgggagctactatgggacagttccca

SEQ ID NO: 4 - DNA, 28 bp, Artificial Sequence
  Primer Oligonucleotide
taagtatgttgaagaaagattattgtag

SEQ ID NO: 5 - DNA, 28 bp, Artificial Sequence
  Primer Oligonucleotide
taaaaactatcccataataactcccaac

SEQ ID NO: 6 - DNA, 633 bp, Artificial Sequence
  Amplification Product Of Bisulfite-Treated DNA
taagtatgttgaagaaagattattgtagaaaaattttttttagtttttttaaaggtgttaggaagtagaaaggtgatatagaattggagaggtcggagtttttgtattaattgtattaaatgcgaatttcgagaaaattttttttaattacgttttgtagttatatggatatgaagatttatgtgaattttgaaagacgtgtttatataagttgaaatgtttttaatgatttagttgatgcgcgtttttttatttgttttttttagagaggtgtaacggaagttagaatattttttttggaaatttaatttgtttcgtagtttttcgaggaattagtatttagttaattcgggtcgggagtagttatttgtggtgaggttgattggttgggtaggaatagcgtcggggcgtgggttgagtatagtcgtttcgttttttttgttataggaagtttgagtttattcgagtagcggtttttttaagtttaaagaagtagaggtcgttgttcgttttttttaggtttttttattaaagtcggagtatttttttttaaaattttacgttttggtggtcgttttaaggagcgcgaggtaggggtacgtaaagttgggagttattatgggatagttttta

SEQ ID NO: 7 - DNA, 686 bp, Artificial Sequence
  Amplification Product Of NME3 Gene
aagggaataaagagaaaagaagtacccagggtcgtggtgtctttgcgctctgtctttaggaccggggagagaagggctgacgctgtggtcgtggccctggccgggggggcgcggggggggcggggttcgggcggtgcggagcagggcgccgcgtgggtggaaccacctgggcgggttgtgggggatacagttagtgtccgagctgctggaggagacttggcctccgcagctgccctccggccccccacggctgccgggttccggggtgcaagtgaagcagcctccccgcggaggccgcagcgccccgaccaggcctctttaagcgcaggccccgccccgggcgccaccgccccgccccgcggatcccgctcccgcaccgccatcatgatctgcctggtgctgaccatcttcgctaacctcttccccgcgggtgagccgcgcggcgcgggccgggggcgggtggccggtgctgggccggcctgacggcccgtccccgcctgccccgcagcctgcaccggcgcacacgaacgcaccttcctggccgtgaagccggacggcgtgcagcggcggctggtgggcgagattgtgcggcgcttcgagaggaagggcttcaagttggtggcgctgaagctggtgcaggtgggggcgcggtgagcgagcgggggcgcggtgtggggggaagggga

SEQ ID NO: 8 - DNA, 24 bp, Artificial Sequence
  Primer Oligonucleotide
aagggaataaagagaaaagaagta

SEQ ID NO: 9 - DNA, 16 bp, Artificial Sequence
  Primer Oligonucleotide
tccccttccccccaca

SEQ ID NO: 10 - DNA, 686 bp, Artificial Sequence
  Amplification Product Of Bisulfite-Treated DNA
aagggaataaagagaaaagaagtatttagggtcgtggtgtttttgcgttttgtttttaggatcggggagagaagggttgacgttgtggtcgtggttttggtcgggggggcgcggggggggcggggttcgggcggtgcggagtagggcgtcgcgtgggtggaattatttgggcgggttgtgggggatatagttagtgttcgagttgttggaggagatttggttttcgtagttgtttttcggttttttacggttgtcgggtttcggggtgtaagtgaagtagttttttcgcggaggtcgtagcgtttcgattaggtttttttaagcgtaggtttcgtttcgggcgttatcgtttcgtttcgcggatttcgttttcgtatcgttattatgatttgtttggtgttgattattttcgttaatttttttttcgcgggtgagtcgcgcggcgcgggtcgggggcgggtggtcggtgttgggtcggtttgacggttcgttttcgtttgtttcgtagtttgtatcggcgtatacgaacgtatttttttggtcgtgaagtcggacggcgtgtagcggcggttggtgggcgagattgtgcggcgtttcgagaggaagggttttaagttggtggcgttgaagttggtgtaggtgggggcgcggtgagcgagcgggggcgcggtgtggggggaagggga